(19) United States
(12) Patent Application Publication
(10) Pub. No.: US 2016/0296376 A1
(43) Pub. Date: Oct. 13, 2016

(54) CONTROL DEVICE AND METHOD FOR CALIBRATING A LASER SYSTEM

(71) Applicant: CARL ZEISS MEDITEC AG, Jena (DE)

(72) Inventors: Michael Stefan Rill, Jena (DE); Tobias Damm, Munich (DE); Delbert Peter Andrews, Oberkochen (DE)

(21) Appl. No.: 15/025,596

(22) PCT Filed: Sep. 30, 2014
(86) PCT No.: PCT/EP2014/002661

§ 371 Date: Mar. 29, 2016

(30) Foreign Application Priority Data
Sep. 30, 2013 (DE) 10 2013 016 336.6

(51) Int. Cl. A61F 9/008; A61B 34/20

(52) U.S. Cl. CPC A61F 9/00836; A61B 34/20; A61F 2009/00855

(57) ABSTRACT

A control device for performing relative calibration of a position of a focal point of a laser, the laser being configured to introduce cuts into tissue. The control device is configured to capture the actual geometry or the actual position of a calibration cut present in the tissue and calibrate the position of the focal point on the basis of the actual geometry or the actual position of the calibration cut.

### CROSS REFERENCE TO RELATED APPLICATIONS

This application is a U.S. National Stage Application under 35 U.S.C. §371 of International Application No. PCT/EP2014/002661 filed on Sep. 30, 2014, and claims benefit to German Patent Application No. DE 10 2013 016 336.6 filed on Sep. 30, 2013. The International Application was published in German on Apr. 2, 2015 as WO 2015/043771 A1 under PCT Article 21(2).

### FIELD

The invention relates to a control device and a method for the relative calibration of the position of a focal point of a laser for introducing cuts into tissue, in particular into the lens of a human or animal eye.

### BACKGROUND

In laser medicine, a highly precise alignment of a laser in relation to an organ or tissue of a patient is usually necessary in order to be able to carry out therapy or tissue processing with the laser in an entirely targeted manner in particular areas of the tissue. In connection with the treatment of the human or animal eye, femto- or nanosecond lasers are often used which can cut or ablate tissue. These lasers must be positioned as precisely as possible relative to the eye or to the tissue of the eye. A laser beam can be focused into predetermined points only in the case of a known absolute and relative arrangement. However, before the application of a laser beam, there is usually at best an insufficient calibration using any reference surface. When contact lenses or other instruments which are made to rest against the eye are used, it usually cannot be avoided that the eye deforms in some way and in particular that the lens of the eye is shifted. A laser processing of particular tissue areas is hereby made more difficult. In particular, the position of the cuts to be introduced into the lens can then often no longer be predetermined with satisfactory precision.

### SUMMARY

In an embodiment, the present invention provides a control device for performing relative calibration of a position of a focal point of a laser, the laser configured to introduce cuts into tissue, wherein the control device is configured to capture the actual geometry or the actual position of a calibration cut present in the tissue; and calibrate the position of the focal point on the basis of the actual geometry or the actual position of the calibration cut.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1a is a schematic side view of a calibration arrangement for a method for absolute calibration according to an embodiment of the invention;

FIG. 1b is a schematic top view of a a test body into which a test cut is introduced which can be measured with several tomographic B-scans according to a method for absolute calibration of an embodiment of the invention;

FIG. 2 is a schematic section view of a human eye against which a patient interface is made to rest, wherein for the purpose of relative calibration in conjunction with a method according to an embodiment of the invention a cut has been introduced into a lens of the eye;

FIG. 3 is a schematic representation of individual steps of a method for relative calibration according to an embodiment of the invention;

FIG. 4 is a schematic representation of individual steps of a method for absolute calibration according to an embodiment of the invention; and

FIG. 5 is a schematic representation of a calibration arrangement which is set up for an absolute calibration and which can be used in conjunction with the relative calibration according to an embodiment of the invention.

### DETAILED DESCRIPTION

According to an embodiment, the invention provides a device or a method with which a precise calibration of a laser can be ensured. According to an embodiment, a control device is provided for the relative calibration of the position of a focal point of a laser for introducing cuts into tissue, wherein the control device is set up to capture the actual geometry and/or the actual position of a calibration cut present in the tissue. In addition, the control device is set up to calibrate the position of the focal point on the basis of the actual geometry and/or the actual position of the calibration cut. A calibration specifically in the tissue area to be treated can hereby be effected precisely.

The control device is set up to carry out the steps of a method according to an embodiment of the invention. In particular, according to a preferred embodiment, the control device has a memory with stored theoretical geometry data or theoretical position data of the calibration cut and is set up to carry out the calibration by correlating the actual geometry and/or actual position with the theoretical geometry and/or theoretical position. Different geometries of the calibration cut can hereby be called on. The geometry of the calibration cut can be selected specifically in relation to the size and geometry of the tissue area to be processed, which can ensure a good precision of the calibration.

According to a preferred embodiment, the control device is set up to carry out the calibration in relation to specific areas of the tissue, in particular by examining the calibration cut in the respective areas of the tissue, or by evaluating the data of the calibration cut in the respective areas of the tissue. A laser can hereby be calibrated such that e.g. a lateral movement of the focal point is effected particularly precisely.

According to a preferred embodiment, the control device is set up to carry out the calibration in relation to all three spatial directions, in particular by evaluating data of the calibration cut in all three spatial directions, wherein the calibration cut has a three-dimensional geometry, or has a two-dimensional geometry extending in all possible directions of movement of the focal point. A laser can hereby be calibrated such that a volumetric processing of tissue is effected precisely.

The control device is preferably connected to the laser and the optics device and has an arithmetic unit and a memory. The control device is set up to compare the actual position data captured by the optics device with theoretical position data of the calibration cut and, if there is a difference between them, to calibrate the position of the focal point of the laser or at least to provide a correction of the position of the focal point. The focal point can then be readjusted either automatically by means of the control device or manually on the laser. The control device can feature or comprise the optics device.

According to an embodiment, a method is provided for the relative calibration of the position of a focal point of a laser for introducing cuts into tissue, in particular into the lens of a human or animal eye, with the steps of:
capturing the actual geometry and/or the actual position of a calibration cut which can be produced by a movement or shift of the focal point; and
calibrating the position of the focal point on the basis of the actual geometry and/or the actual position of the calibration cut;
wherein the calibration cut inside the tissue is captured by examining the tissue.
   The calibration can comprise correcting the position of the focal point. It has been shown that a particularly precise calibration can be effected using a calibration cut in tissue which, during or after a treatment, is removed and is no longer needed, or which is to be replaced. The calibration can be effected in relation to those areas which are to be reached by the laser. The calibration cut is preferably measured precisely in those tissue areas in which the laser is later to carry out a processing of the tissue (e.g. a fractionation).

In contrast, an absolute calibration of the system is usually very expensive. It would therefore previously usually only have been carried out once, on delivery of a respective laser system or optics system. In the case of an absolute calibration, the distances and geometries between an optics system and a laser system can be defined. Relative calibration on the other hand can be effected in a relatively short time. This was previously able to take place e.g. within the framework of a self-test of the system on start-up of the system. The present invention on the other hand is based on the knowledge that during laser therapy the patient's eye also forms part of the whole optical system, which cannot yet be taken into account during the production and setting-up of a laser system. By way of the relative calibration according to an embodiment of the invention, there is now the possibility of taking into account patient-specific optical properties of the respective eye (e.g. the refractive power of the cornea, optical aberrations in the eye, etc.), and adapting the laser system to these. A focal point of a laser can thereby be positioned and moved more precisely relative to the tissue of the patient's eye.

A relative calibration immediately before a treatment or before an intervention and specifically in relation to the tissue areas to be treated provides a high precision. An absolute calibration alone is usually not sufficient to be able to ensure a sufficiently high precision. This is because, when a patient interface is used, e.g. a contact lens, or generally in conjunction with a therapy or treatment, pressure is exerted on the eye of a patient, and the lens moves in some way inside the eye. Even if a patient's eye was measured in the unloaded state and all details of the geometry and arrangement of the lens are known, a (re-)calibration is usually necessary as soon as a patient interface, in particular a contact lens, is made to rest against the eye with a particular pressure and the pressure ratios in the eye change and the lens assumes another position. By a patient interface can be meant a contact lens or e.g. a funnel-shaped patient docking system. A fluid (preferably saline solution) can be poured into the funnel-shaped patient docking system. To connect the patient docking system to the laser system, an end lens of the laser system can be immersed in the fluid, in particular to adapt the refractive index.

A relative calibration, in particular immediately before a therapeutic intervention, brings the advantage that a higher cut precision of the laser can be achieved, in particular because optical peculiarities, e.g. the lens geometry, in the respective eye of a patient can be taken into account, i.e. also differences between the two eyes of a patient. The introduction of laser cuts in selective areas of the lens, e.g. only in a segment of a nucleus of the lens, can hereby be effected even more precisely, e.g. inside the lens. The relative calibration according to an embodiment of the invention can be carried out e.g. immediately before a laser-supported cataract operation.

Owing to the relative calibration, a precise control of the laser can be effected, in particular on the basis of image data captured by means of an optics device. The arrangement or position of the focal point of the laser can be made to relate to the optics device, and a control of the laser, in particular the positioning and moving of a focal point of the laser, can be effected particularly precisely on the basis of image data which are produced by the optics device. Through the absolute calibration, the system integration, i.e. the match between optics device and laser, can be effected particularly precisely. The optics device is set up to examine the tissue.

The calibration cut has a definable geometry, in particular a circular or linear geometry. The calibration cut can be produced by moving a focal point of the laser in the tissue, wherein a multi-photon absorption is preferably effected in a laser focal volume. The calibration cut can also be at least approximately punctiform, but it preferably extends at least two-dimensionally, in particular in order to be able to locate the calibration cut easily by means of individual scans (sectional images through the tissue), and to be able to carry out a calibration in relation to several spatial directions.

The laser is preferably arranged in a definable position relative to the optics device, in particular an optical coherence tomograph (OCT). The optics device is preferably an OCT, but it can also in particular be another type of tomographic optics device, e.g. also a Scheimpflug camera. This also applies correspondingly to an absolute calibration, as explained in even more detail elsewhere.

The actual position of the calibration cut relative to the optics device is preferably captured. The actual geometry and the actual position are preferably captured optically in each case by means of the optics device. In other words, the examination of the tissue can comprise a transillumination or scanning of the tissue. The examination is preferably effected by scanning of the tissue in those areas which are to be treated with a subsequent medical procedure.

By calibration or calibrating is preferably meant establishing and taking into account a deviation of the actual position of the focal point from a theoretical position of the focal point, in particular on the basis of position data which describe the geometry and arrangement of the calibration cut.

According to a preferred embodiment, the calibration comprises comparing the captured actual geometry and/or actual position with a theoretical geometry and/or theoretical position preferably stored in a memory. The theoretical/actual comparison of a particular geometry brings the advantage that the geometry can be matched to the laser cuts to be carried out. This makes a high precision of the calibration possible, especially with regard to the laser cuts to be carried out.

The theoretical geometry preferably corresponds to a geometry which was transmitted to the laser. According to a variant, the theoretical geometry is stored in a memory of the optics device, such that the memory of the optics device can be accessed during the comparison.

According to a preferred embodiment, the calibration is effected on the basis of the actual position relative to a reference mark or reference surface, in particular a reference surface or mark of a patient interface (e.g. contact lens), as well as on the basis of the position of the laser relative to the optics device. The reference surface is preferably defined by the optics apparatus itself. The reference surface particularly preferably lies in the optics apparatus. The calibration can hereby be effected independently of a respective position of a patient. The position of the reference surface relative to the laser is known, and the arrangement of the laser relative to the optics device is also known. Thus, only the arrangement of the calibration cut relative to the reference surface has to be determined. The reference mark can be used for the absolute local alignment of laser system, optics system and patient's eye.

According to a variant, a patient interface is provided with a reference mark or reference surface. The reference surface is arranged in a definable position relative to the optics device and in a definable position relative to the tissue, wherein the capture of the actual position and/or actual geometry is effected through the patient interface.

If a patient interface is used, the calibration method according to an embodiment of the invention can be developed by further steps, in particular by the steps of:
1) providing a patient interface with a reference mark or reference surface on the cornea of the eye;
2) determining an axial distance between a front side of the eye lens and the reference surface by means of the optics device, in particular by means of an OCT system;
3) determining an axial distance between a rear side of the lens and the reference surface by means of the optics device;
4b) capturing the actual geometry of the calibration cut by means of the optics device;
5) comparing the captured actual geometry of the calibration cut with a theoretical geometry stored in a memory, and
6) calibrating the position of the focal point of the laser on the basis of the position of the calibration cut relative to the reference surface as well as on the basis of the position of the laser relative to the optics device.
   The distance determination of the front and the rear side of the lens can in particular be used for the secure positioning of the focal point inside the lens tissue and not too close to the edge of the lens. The distance determination of the front and the rear side of the lens can also be checked repeatedly during the laser cutting, e.g. in order to detect lens shifts and to set the position of the focal point accordingly.

Before step 4b), in a method step separate from the calibration method, in a step 4a), the calibration cut can be introduced between the front side and the rear side. This step is not a method step of the calibration method according to an embodiment of the invention. The calibration method only comprises capturing the position of the lens and predetermining an area in which a calibration cut can be introduced, but not the introduction of the calibration cut. The introduction of the calibration cut is a step, in particular a surgical intervention, which is carried out in a method step independent of the calibration method. The calibration method only comprises measuring the introduced, already present calibration cut.

According to a preferred embodiment, a patient interface, in particular a contact lens, is arranged between the tissue and the optics device, and the capture of the actual position and/or actual geometry is effected through the patient interface. The relative calibration can hereby be effected under conditions under which a therapy or treatment can subsequently be effected. By means of the patient interface, the arrangement of the components relative to each other in particular can be defined, and the pressure ratios in the eye and the location of the lens in the eye can be kept within definable, narrow value ranges.

According to a preferred embodiment, the examination comprises acquiring at least two sectional images (so-called B-scans) cutting through the tissue in different planes. The calibration cut can hereby be captured with good precision largely independently of its real location and alignment, in particular without a plurality of sectional images having to be acquired in order to initially locate the calibration cut.

The geometry of the calibration cut is preferably captured by acquiring at least two B-scans cutting through the calibration cut, wherein the B-scans are preferably arranged at an angle relative to each other in the range of from 30 to 90 degrees, in particular 90 degrees.

According to an embodiment, the invention provides a computer program product having computer code set up to carry out a method on a computer or arithmetic unit, in particular an arithmetic unit of the control device.

A method according to an embodiment of the invention for relative calibration can also be carried out directly in conjunction with a method step for introducing a calibration cut. Such a method for the relative calibration of the position of a focal point of a laser for introducing cuts into the lens of a human or animal eye preferably comprises the steps of:
introducing a calibration cut with a definable geometry, in particular a circular or linear geometry, by means of the laser into the eye lens by moving the focal point inside the lens;
capturing the actual geometry of the calibration cut present in the lens and/or the actual position of the calibration cut; and
calibrating the location of the focal point of the laser on the basis of the actual geometry and/or the actual position of the calibration cut; wherein the calibration cut inside the tissue is captured by examining the tissue.
   In other words, the method according to an embodiment of the invention for relative calibration can be supplemented by a method step of introducing a calibration cut with a definable geometry. The calibration cut is preferably introduced during a preoperative planning phase. The calibration cut is preferably introduced into tissue which is removed and replaced during a subsequent operation, with the result that no side effects develop for the patient. The calibration cut is preferably introduced into that area or section of the lens which is to be treated in a subsequent treatment step by means of the laser. The laser treatment can hereby be effected particularly precisely. For example, a fine fragmentation of a particular segment of the lens can be effected without the danger of damaging the capsular bag.

Before or after the method for relative calibration, a method for absolute calibration can be carried out. The precision can be further improved by an absolute calibration. The absolute calibration can be carried out e.g. after transporting the laser or after a certain amount of time, or also after a significant temperature or pressure fluctuation (in particular in the case of a different usage site).

A method for the absolute calibration of a laser for introducing cuts into tissue, in particular into the lens of a human or animal eye, wherein the laser is arranged in a definable position relative to the optics device, in particular an OCT camera, preferably has the following steps of:
a) providing a test body as well as a patient interface with a reference surface and with a reference point arranged on the reference surface;
b) arranging the patient interface in a definable position relative to the test body, in particular connecting the patient interface to the test body, and positioning the reference surface relative to the optics device;
c) introducing a test cut with a definable geometry, in particular a circular geometry, and known dimensions, and at a known axial distance from the reference surface, into the test body by means of laser radiation of the laser focused into a focal point;
d) scanning the test body at least in the area of the test cut by means of the optics device, wherein at least two sectional images are acquired, in particular B-scans are carried out, which are preferably arranged at an angle and at a lateral distance relative to each other, wherein at least two points of intersection of the test cut with the sectional images are preferably ascertained;
e) determining an axial distance and a lateral distance of at least one point of intersection of the test cut with the respective sectional image relative to the reference point; and
f) calibrating the position of the focal point of the laser, in particular on the basis of the position of the laser relative to the optics device.
   Step f) can comprise determining the position of the focal point. Preferably, a lateral position of the focal point is calibrated on the basis of the lateral distance and an axial position of the focal point is calibrated on the basis of the axial distance.

A first calibration or also a recalibration can be effected by means of the method for absolute calibration. The calibration can be carried out quickly and easily, in particular because of the clear arrangement of the optics device relative to the laser and relative to the patient interface. The absolute calibration can also be carried out immediately before a therapy or treatment, in particular by means of the same or a new test body. Calibration data obtained by the calibration can be used to carry out a control of the laser and/or the optics device by means of a computer program.

The term “B-scan” can refer to a scanning in a plane which cuts through the test body, in particular in the direction of view or a laser irradiation direction. A B-scan is preferably composed of a plurality of linear A-scans. In contrast to an A-scan extending along one axis, a B-scan comprises image data of a whole plane. Alternatively, the test body can be scanned by means of the optics device before the introduction of the cut.

The term “a definable position” can refer to a defined (predetermined) position, and thus one known to the whole system. It can be a specific predefined position, or the position is measured and ascertained beforehand. By a definable geometry is meant a geometry which can be predetermined and is exactly known, e.g. a strictly circular shape with a known diameter and known alignment in space, or an ellipse with known main axes.

The position of the focal point can be determined on the basis of the axial distance and the lateral distance, and on the basis of the arrangement of the laser relative to the patient interface, in order then to calibrate the position of the focal point. The arrangement of the sectional images relative to the reference point is previously known or definable.

In a further step g) an absolute measurement of the geometry of the cut and of the lateral or axial distance of the cut from the reference surface can then be effected. Step g) can precede step f). The test body can be opened, and the lateral distance and the axial distance can be determined absolutely.

The test cut introduced (cut) by the laser is e.g. a closed line, in particular a circle. Alternatively, another geometry that extends at least two-dimensionally, e.g. a cross, can be used. Alternatively, several structures, in particular several circles or ellipses, or e.g. also a grid, can be introduced. Optical imaging errors of the laser optics can be determined hereby, with the result that measures can be taken to compensate for the imaging errors. Imaging errors are characterized in that the real pattern of shift of the laser beam deviates from a predetermined strict geometric pattern.

A laser which can cut transparent media on the basis of multi-photon absorption is preferably used. For example, gas lasers, dye lasers, solid-state lasers or semiconductor lasers can be used as the laser. Variations are also possible for the type of optical capture, in particular the type of tomographic imaging, e.g. depth scans or any type of 3D imaging.

A method according to an embodiment of the invention for calibrating a laser can comprise the method steps of the relative calibration method as well as the method steps of the absolute calibration method. The method for absolute calibration can be carried out by means of a calibration arrangement, in particular a calibration arrangement for the absolute calibration of a laser for introducing cuts into tissue, in particular into the lens of a human or animal eye, with:
a test body, in particular a Plexiglas sphere which is transparent for laser radiation of the laser and which can be arranged in a definable position relative to a patient interface, in particular resting against the patient interface;
the patient interface with a reference mark or reference surface and a reference point, arranged on the reference surface, which can be arranged in a definable position relative to the test body;
wherein the test body is designed such that the geometry and arrangement of a test cut introduced into the test body can be captured in absolute values, in particular also the arrangement of the test cut relative to a surface of the test body, against which the patient interface can be made to rest.
   The test body can be provided for single or multiple use. In the case of multiple use, the test cuts are then in each case to be introduced in different positions and preferably not overlapping, thus at a distance from each other.

The calibration arrangement preferably furthermore comprises the laser, which is set up to introduce a test cut into the test body; and an optics device (e.g. OCT system or Scheimpflug camera) arranged in a defined position relative to the laser with a measuring apparatus which is set up to determine the position of the test cut, and thus the position of a focal point of the laser, relative to the reference surface.

The laser can be arranged between the optics device and a surgical microscope, wherein the optics device can also be integrated in the surgical microscope. The laser is preferably arranged in a definable position relative to the optics device. The actual position is preferably captured relative to the optics device. The actual position and the actual geometry are preferably captured in each case by means of the optics device.

The test body is preferably a body which can be exposed in the area of the test cut such that the arrangement of the test cut, in particular a lateral distance and an axial distance of the test cut from a reference point, can be determined absolutely. The material of the test body can be chosen depending on the type of laser such that the laser is set up to introduce a test cut into the test body. A material which matches the optical properties of the human or animal eye tissue as closely as possible, in particular a plastic, preferably Plexiglas, is preferably chosen. The shape and size of the structure cut with the laser can be chosen such that a point of intersection of an auxiliary line with the structure can be determined easily. The test body is preferably adapted to the given application and set up to reproduce or image the optical and geometric properties of the tissue to be treated as precisely as possible.

The reference mark or reference surface is preferably arranged, together with the reference point, on a surface of the patient interface, which is set up to come to rest against the test body. In other words, the test body preferably has a bearing surface which is formed to correspond geometrically to a corresponding surface of the patient interface. The absolute measurement of the test cut on the test body can hereby be simplified, in particular because the measurement can be effected in relation to a surface of the test body.

A cut into the test body can be referred to as “a test cut,” for the purpose of better differentiation, and a cut into tissue or into the lens can be referred to as “a calibration cut.” The test body can also be provided with markers which are in communication with the laser, wherein by means of the markers the movement of the optics device, or the position of the laser focal point, or an alignment of individual components relative to each other (alignment), can be reconstructed. The markers are implemented beforehand, thus their location relative to each other and their position on the test body is known. For example corner points of a cubic or hexagonal grid which are arranged in a definable position with known lateral and axial position data come into consideration as markers. The control apparatus can be set up to move the focal point such that all grid points are connected to each other. If the laser has not been calibrated sufficiently precisely, this can be recognized by the fact that the focal point has not been/cannot be positioned in some grid points. This can be evaluated by means of the optics device.

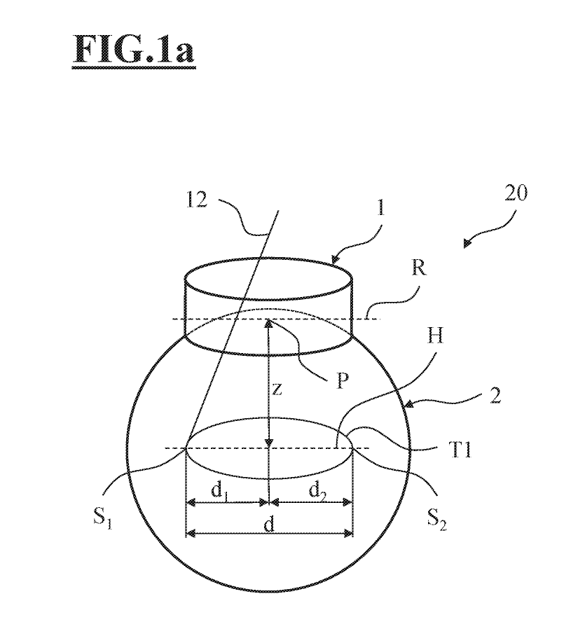

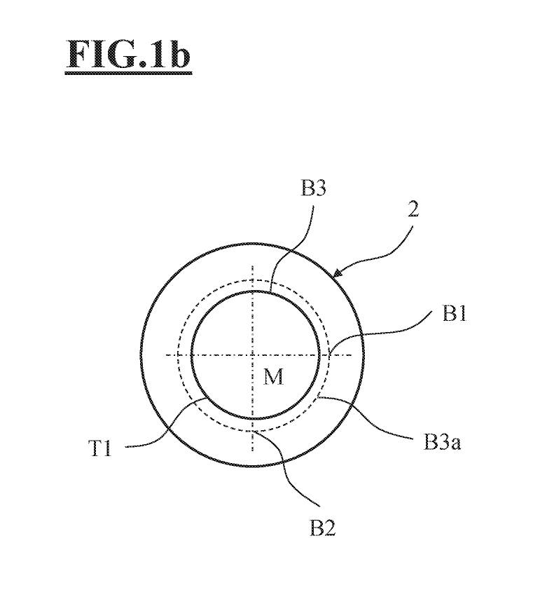

A calibration arrangement 20 for an absolute calibration is shown in FIG. 1a, with a patient interface in the form of a contact lens 1 which rests or has been made to rest against a test body 2. A laser beam 12 of a laser (not represented) runs though the contact lens 1 and is focused in a focal point into the test body 2 and cuts a test cut T1 (here in the shape of a circle) into the test body 2 by moving the focal point. The contact lens 1 has a reference surface R against which the test body 2 comes to rest. A reference point P is arranged on the reference surface R. The test cut T1 has a circular shape with a diameter d and is arranged at an axial distance z from the reference surface R of the contact lens 1. In the area of the test cut T1 an auxiliary line H (in particular for the purpose of better understanding) is indicated which cuts through the test cut T1 in two intersection points S1, S2. The test body 2 was transilluminated or examined by an optics device (not represented) in at least two scans or section planes (B-scans), and the intersection points S1 and S2 were determined. The first intersection point S1 is arranged at a lateral distance d1 from the reference point P, and the second intersection point S2 is arranged at a lateral distance d2 from the reference point P. The auxiliary line H is indicated in order to emphasize the respective intersection point S1, S2 of the B-scan with the test cut T1. Via the distances z, d1, d2 the geometry and arrangement of the test cut T1 can be determined in order to ascertain from this the setting of the laser and, if necessary, to calibrate the laser.

In FIG. 1b a test body 2 into which a circular test cut T1 has been introduced is shown in top view, and the test cut T1 is arranged concentrically around a central point M, wherein the central point M can also correspond to a central point of the test body 2. The test body 2 is measured using several B-scans, namely a first B-scan B1 and a second B-scan B2, which both run through the central point M, as well as a ring scan B3 which extends along the test cut T1 or overlaps the test cut T1, i.e. cuts through the test cut T1. By means of these three scans the test body 2 (or alternatively a lens) and the test cut T1 can be geometrically captured easily and precisely. The two flat B-scans are preferably not aligned parallel to each other. According to a variant the two flat B-scans can, as represented, be aligned at least approximately orthogonal to each other. Alternatively, further flat scans or further ring scans can be effected, as indicated by the further ring scan B3a, in particular if this is expedient for a particularly high precision. A B-scan is to be understood as a type of cross section through the test body 2 or through a lens and is composed of a plurality of purely axial measurements (A-scans). The test cut T1 can also have a geometry deviating from the circular shape, but it has been shown that a calibration on the basis of the circular shape is particularly expedient, in particular because a large part of the positions which are to be/have to be traced by the focal point can be captured. This makes calibration easier. The calibration can be adapted specifically to the positions to be traced.

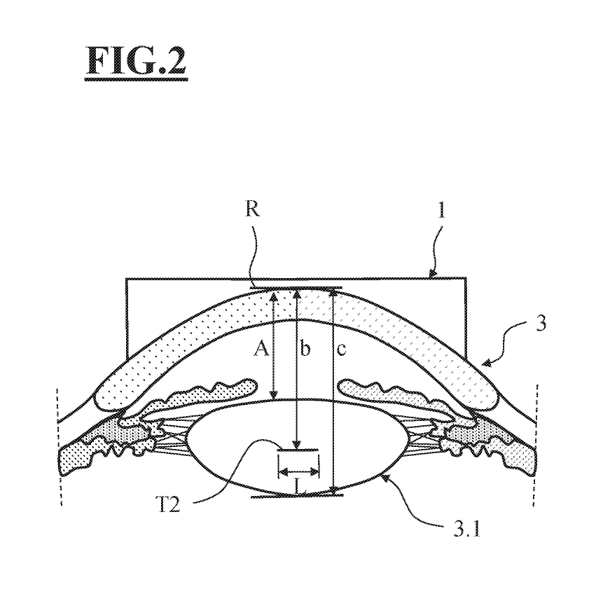

An arrangement is schematically shown in FIG. 2 by means of which a relative calibration of an imaging optics device (not represented), in particular an optical tomography device (e.g. OCT or Scheimpflug camera), can be effected. A patient interface in the form of a contact lens 1 is arranged on the cornea of an eye 3 and has a reference surface R. The reference surface R is arranged at an axial distance A from a front side of a lens 3.1 of the eye 3. The reference surface R is arranged at an axial distance C from a rear side of the lens 3.1. Inside the lens 3.1 a calibration cut T2 is introduced by means of a laser (not represented). The calibration cut T2 has a known, predeterminable, two- or three-dimensional geometry, e.g. a linear shape or a circular shape with predefined dimensions. In the example represented, the calibration cut T2 is linear and has the length L. The calibration cut T2 is arranged at a known, predeterminable axial distance B (depth in relation to the reference surface) from the reference surface R, inside the lens 3.1.

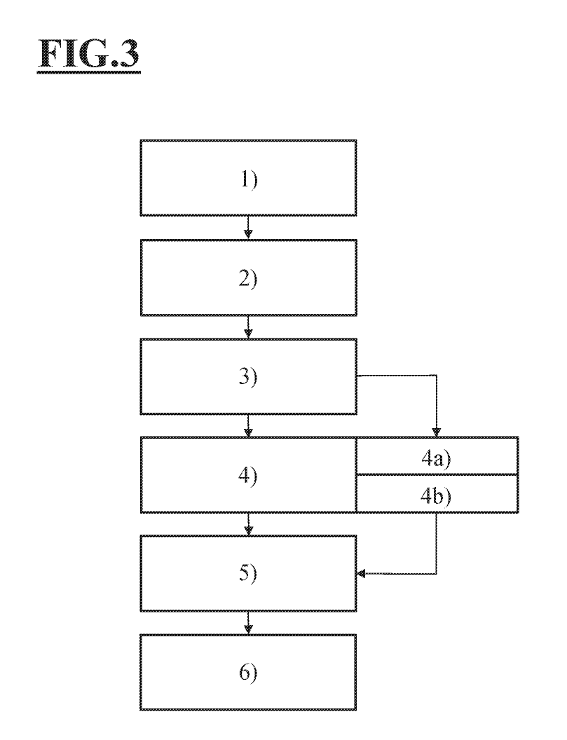

A method for the relative calibration of a laser is shown in FIG. 3, with the steps of
1) providing a patient interface with a reference mark or reference surface and arranging the reference mark or reference surface in a definable position relative to the optics device and in a definable position relative to the tissue;
2) determining an axial distance between a front side of the tissue and the reference mark or reference surface by means of the optics device;
3) determining an axial distance between a rear side of the tissue and the reference mark or reference surface by means of the optics device;
4) capturing the actual geometry of a calibration cut present in the tissue by means of the optics device, wherein the calibration cut has a definable geometry, in particular a circular or linear geometry, and wherein the calibration cut can be produced by moving a focal point of the laser;
5) comparing the captured actual geometry of the calibration cut with a theoretical geometry stored in a memory; and
6) calibrating the position of the focal point of the laser on the basis of the actual geometry of the calibration cut, in particular on the basis of the position of the calibration cut relative to the reference surface as well as on the basis of the position of the laser relative to the optics device.

Step 4) can be divided into steps 4a) and 4b), with 4a) being to introduce the calibration cut between the front side and the rear side; and

4b) being to capture the actual geometry of the calibration cut by means of the optics device. However, the calibration method according to an embodiment of the invention only comprises capturing the calibration cut, not introducing the calibration cut. The calibration cut is preferably introduced in an area of the tissue which is to be treated within the framework of a subsequent medical procedure.

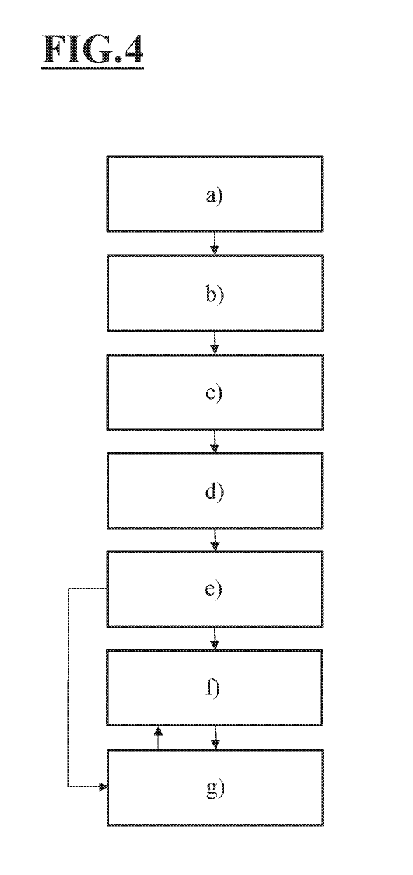

A method for the absolute calibration of a laser is shown in FIG. 4, with the steps of
a) providing a test body as well as a contact lens with a reference surface and with a reference point arranged on the reference surface;
b) arranging the contact lens in a definable position relative to the test body, in particular connecting the contact lens to the test body, and positioning the reference surface relative to the optics device;
c) introducing a test cut with a definable geometry, in particular a circular geometry, and known dimensions, and at a known axial distance from the reference surface, into the test body by means of laser radiation of the laser focused into a focal point;
d) scanning the test body at least in the area of the test cut by means of the optics device, wherein at least two sectional images are acquired, in particular B-scans are carried out, which are preferably arranged at an angle and at a lateral distance relative to each other, wherein at least two points of intersection of the test cut with the sectional images are preferably ascertained;
e) determining an axial distance and a lateral distance of at least one point of intersection of the test cut with the respective sectional image relative to the reference point; and
f) calibrating the position of the focal point of the laser, in particular on the basis of the position of the laser relative to the optics device. Preferably, a lateral position of the focal point is calibrated on the basis of the lateral distance and an axial position of the focal point is calibrated on the basis of the axial distance. In a further step g) an absolute measurement of the geometry of the cut and of the lateral or axial distance of the cut from the reference surface can then be effected. Step g) can precede step f). The test body can be opened, and the lateral distance and the axial distance can be determined absolutely.

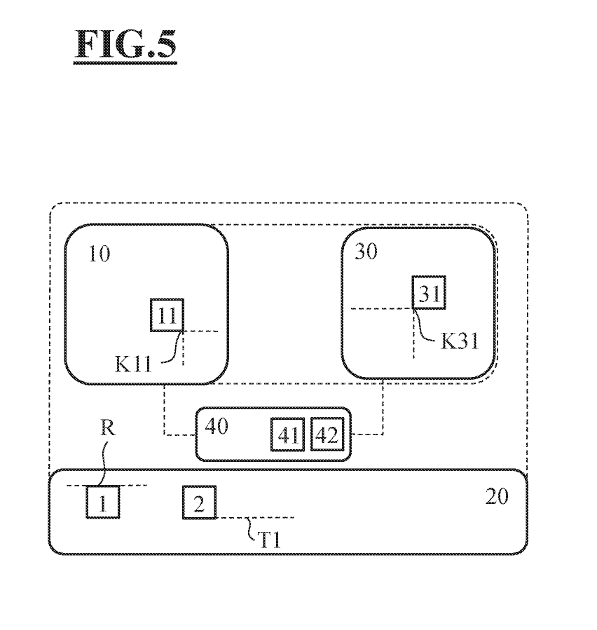

In FIG. 5 the calibration arrangement 20 is shown schematically in conjunction with the laser system 10 and the optics device 30 as well as a control device 40, wherein the laser system 10 has a laser 11 which has a reference coordinate K11 with a known position, and wherein the optics device 30 has a measuring apparatus 31 (in particular an interference structure with a low-coherence light source or a Scheimpflug arrangement) which has a reference coordinate K31 with a known position.

The calibration arrangement 20 has the contact lens 1 and the test body 2, wherein the contact lens 1 with the reference surface R is arranged relative to the test body 2. According to a variant the contact lens 1 is connected to the test body 2 or is found in direct contact therewith and rests with the reference surface R against a surface of the test body 2. The test cut T1 is introduced into the test body 2.

The calibration arrangement 20 can, depending on the definition, also comprise the laser system 10 and/or the optics device 30 or in each case at least one component thereof. The laser system 10 can, depending on the definition, also comprise the optics device 30 or at least one component thereof.

The control device 40 is connected to the laser system 10 and/or the optics device 30 and is set up to control the laser system 10 and the optics device 30. The control device 40 has a memory 41 in which theoretical geometries or theoretical positions for calibration cuts are stored. The control device 40 has an arithmetic unit 42. The control device 40 is set up to capture the position of the laser system 10 relative to the optics device 30 and to the contact lens 1 and to take this into account during a correlation of actual and theoretical data.

The method according to an embodiment of the invention for relative calibration makes it possible to calibrate, immediately before a treatment, the position of the focal point specifically in relation to tissue areas which are to be processed, in particular removed, in a subsequent treatment step. A calibration cut introduced into the relevant tissue area is also examined in the relevant tissue area, whereby a high precision of the calibration can be ensured.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMBERS

1 Patient interface, in particular contact lens
2 Test body, e.g. Plexiglas sphere
3 Eye
3.1 Lens
10 Laser system
11 Laser
12 Laser beam
20 Calibration arrangement
30 Optics device, in particular tomography device, preferably optical coherence tomograph
31 Optical measuring apparatus, in particular OCT laser
40 Control device
41 Memory
42 Arithmetic unit or computer
A Tissue front side, in particular front side of the eye lens
b Depth or axial distance of the circle described by the laser beam
B1 First B-scan
B2 Second B-scan
B3 Third scan, in particular ring scan
B3a Further ring scan
C Tissue rear side, in particular rear side of the eye lens
d Lateral distance of two ascertained cut positions relative to each other
d1 Lateral distance of a (first) cut position from the reference point
d2 Lateral distance of a second cut position from the reference point
H Auxiliary geometry, in particular auxiliary line
K11 Position of a reference coordinate of the laser
K31 Position of a reference coordinate of the measuring apparatus
L Length or diameter of a cut introduced by the laser beam
M Central point of the test cut
P Reference point
R Reference mark or surface
S1 (First) point of intersection between test cut and auxiliary geometry
S2 Second point of intersection between test cut and auxiliary geometry
T1 Test cut in test body for the purpose of absolute calibration
T2 Calibration cut in lens for the purpose of relative calibration
z Axial distance of the circle described by the laser beam

1. A control device for performing relative calibration of a position of a focal point of a laser, the laser configured to introduce cuts into tissue, wherein the control device is configured to:
capture the actual geometry or the actual position of a calibration cut present in the tissue; and
calibrate the position of the focal point on the basis of the actual geometry or the actual position of the calibration cut.

2. The control device according to claim 1, the control device comprising a memory having stored thereon theoretical geometry data or theoretical position data of the calibration cut, wherein the control device is configured to carry out the calibration by correlating the actual geometry and/or actual position with the theoretical geometry and/or theoretical position.

3. The control device according to claim 1, wherein the control device configured to carry out the calibration in relation to specific areas of the tissue.

4. The control device according to claim 1, wherein the control device is configured to carry out the calibration in relation to three spatial directions.

5. A method for performing relative calibration of a position of a focal point of a laser for introducing cuts into tissue, the method comprising:
capturing an actual geometry and/or an actual position of a calibration cut produced by a movement of the focal point; and
calibrating a position of the focal point on the basis of the actual geometry and/or the actual position of the calibration cut;
wherein the calibration cut inside the tissue is captured by examining the tissue.

6. The method according to claim 5, wherein further comprising comparing the captured actual geometry and/or actual position with a theoretical geometry and/or theoretical position.

7. The method according to claim 5, wherein the calibration is effected on the basis of the actual position relative to a reference surface as well as on the basis of the position of the laser relative to an optics device for examining the tissue.

8. The method according to claim 7, wherein a patient interface is arranged between the tissue and the optics device, and
wherein the capture of the actual position and/or actual geometry is effected through the patient interface.

9. The method according to claim 5, wherein the examination comprises acquiring at least two sectional images cutting through the tissue in different planes.

10. A computer program comprising code having instructions for carrying out a method comprising:
capturing an actual geometry and/or an actual position of a calibration cut produced by a movement of the focal point: and
calibrating a position of the focal point on the basis of the actual geometry and/or the actual position of the calibration cut;
wherein the calibration cut inside the tissue is captured by examining the tissue.
